(19) United States
(12) Patent Application Publication
(10) Pub. No.: US 2026/0103671 A1
(43) Pub. Date: Apr. 16, 2026

(54) Data Processing Apparatus and Methods for Data Processing Using the Same

(71) Applicant: Terumo Kabushiki Kaisha, Tokyo (JP)

(72) Inventors: Nozomu YAMAZAKI, Kanagawa (JP); Masatsugu IGARASHI, Kanagawa (JP)

(21) Appl. No.: 19/418,405

(22) Filed: Dec. 12, 2025

Related U.S. Application Data
(63) Continuation of application No. PCT/JP2024/021740, filed on Jun. 14, 2024.

(30) Foreign Application Priority Data
Jun. 16, 2023 (JP) 2023-098864

(51) Int. Cl. C12M 1/34; C12M 1/26; C12M 1/36

(52) U.S. Cl. CPC C12M 41/36; C12M 33/14; C12M 41/48

(57) ABSTRACT

A data processing apparatus includes an acquisition unit configured to acquire first cell count data under a first culture condition and second cell count data under a second culture condition, a calculation unit configured to calculates a rate ratio that is a ratio between a first change rate of the first cell count data in a first period and a second change rate of the second cell count data in a second period, and a prediction data generation unit configured to generate prediction data that indicates a change in the number of cells after the present time on the basis of first partial data that is a part after the first period of the first cell count data and the rate ratio.

### CROSS-REFERENCE TO RELATED APPLICATIONS

The present application is a continuation of the International Patent Application No. PCT/JP2024/021740 filed June 14, 2024, which designated the U.S. and claims the benefit of priority from Japanese Patent Application No. JP2023098864 filed June 16, 2023. The entire disclosures of the above-identified applications are incorporated herein by reference.

### FIELD

The present invention relates to a data processing apparatus and to methods for data processing using the same.

### BACKGROUND

When using hollow fiber membranes, it is often difficult to directly observe the inside of the hollow fibers during cell culturing processes. There is a need for prediction models for categorizing transition of cell cultures during culturing processes.

JP 2020-171241 A discloses a cell culture apparatus where cells are cultured according to set culture conditions. A user sets a culture condition for the cell culture apparatus such that a desired number of cells can be recovered in a selected culture period.

### SUMMARY

At least one example embodiment relates to an apparatus configured to perform data processing of a cell culture as cultured by a cell culture apparatus that includes a hollow fiber membrane. The data processing apparatus may include an acquisition unit. The acquisition unit may be configured to acquire first cell count data that indicates a change in the number of cells between a first start of a first cell culture using the cell culture apparatus and an end of the first cell culturing under a first culture condition and also second cell count data that indicates a change in the number of cells between a second start of a second cell culture using the cell culture apparatus and present time under a second culture condition. The data processing apparatus may include a calculation unit. The calculation unit may be configured to calculate a rate ratio. The rate ratio may be a ratio between a first change rate and a second change rate, the first change rate being a rate of change of the first cell count data during a first predetermined or selected period, and the second change rate being a rate of change of the second cell count data during a second predetermined or selected period. The second predetermined or selected period may correspond with the first predetermined period. The data processing apparatus may include a prediction data generation unit. The prediction data generation unit may be configured to generate prediction data that indicates a change in the number of cells after the present time of the second culture condition on the basis of first partial data which is a part after the first predetermined period of the first cell count data and the rate ratio. Because the prediction data is a reflection of the second cell count data, which is data during culture, the transition of culture during the cell culture can be predicted and a user of the cell culture apparatus may adapt an initial culture plan on an ongoing basis as needed during cell culture.

In at least one example embodiment, the data processing apparatus may include a display control unit. The display control unit may be configured to control displays on a display unit of the cell culture apparatus. For example, the display control unit may be configured to display on the display unit the number of cells calculated from third cell count data which is data obtained by combining the prediction data with the second cell count data.

In at least one example embodiment, the user may be able to determine a culture period during which a desired number of cells can be recovered based on a number of cells displayed on the display unit.

In at least one example embodiment, the prediction data generation unit may be configured to generate the prediction data when the second change rate deviates from the first change rate, for example, by a predetermined or selected amount.

In at least one example embodiment, the prediction data generation unit may be configured to generate the prediction data only when a difference between the initial culture plan and the actual culture situation exceeds a predetermined or selected threshold (e.g., is larger than the predetermined or selected threshold).

In at least one example embodiment, the first cell count data may include data obtained by previously performed cell cultures, data obtained by a cell culture simulations, or a combination thereof.

In at least one example embodiment, the acquisition unit may be configured to acquire the first cell count data on the basis of a plurality of pieces of cell count data.

At least one example embodiment relates to a method for data processing. The data processing method may include acquiring first cell count data. The first cell count data may indicate a change in the number of cells from a first start to an end of a first cell culture under a first culture condition. The data processing method may include acquiring second cell count data. The second cell count data may indicate a change in the number of cells from a second start of a second cell culture to a present time under a second culture condition. The data processing method may include calculating a rate ratio, where the rate ratio is a ratio between a first change rate and a second change rate. The first change rate may be a rate of change of the first cell count data during a first predetermined or selected period. The second change rate may be a rate of change of the second cell count data during a second predetermined or selected period. The second predetermined or selected period may correspond with the first predetermined period. The data processing method may include generating prediction data. The prediction data may indicate a change in the number of cells after the present time of the second culture condition on the basis of first partial data which is a part after the first predetermined period of the first cell count data and the rate ratio. Because the prediction data is a reflection of the second cell count data, which is data during culture, the transition of culture during the cell culture can be predicted and a user of the cell culture apparatus may adapt an initial culture plan on an ongoing basis as needed during cell culture.

### DRAWINGS

FIG. 1 is a block diagram of an example cell culture system in accordance with at least one example embodiment.

FIG. 2 is an example circuit diagram of an example cell culture circuit of the cell culture system of FIG. 1 in accordance with at least one example embodiment.

FIG. 3 is the circuit diagram of FIG. 2 illustrating where a cell culture apparatus is operated in a first culture form at the time of cell culture in accordance with at least one example embodiment.

FIG. 4 is the circuit diagram of FIG. 2 illustrating where the cell culture apparatus is operated in a second culture form at the time of cell culture in accordance with at least one example embodiment.

FIG. 5 is the circuit diagram of FIG. 2 illustrating where the cell culture apparatus is operated in a third culture form at the time of cell culture in accordance with at least one example embodiment.

FIG. 6 is the circuit diagram of FIG. 2 illustrating where the cell culture apparatus is operated in a fourth culture form at the time of cell culture in accordance with at least one example embodiment.

FIG. 7 is a flowchart illustrating an example method for data display on a cell culture system of FIG. 1 in accordance with at least one example embodiment.

FIG. 8A is a graphical illustration demonstrating first cell count data and second cell count data in accordance with at least one example embodiment.

FIG. 8B is a graph illustrating the first cell count data and third cell count data in accordance with at least one example embodiment.

FIG. 9 is a graphical illustration demonstrating transition data displayed for a display unit of the cell culture system of FIG. 1 in accordance with at least one example embodiment.

FIG. 10 is a table illustrating evaluation results of an example in accordance with at least one example embodiment.

FIG. 11 is a table illustrating a culture condition of each of three specimens in accordance with at least one example embodiment.

FIG. 12 is a graphical illustration demonstrating cell count data (glucose) obtained by simulating a specimen 2 (low) using a specimen 1 (middle) of adherent cells as reference data in accordance with at least one example embodiment.

FIG. 13 is a graphical illustration demonstrating cell count data (lactic acid) obtained by simulating the specimen 2 (low) using the specimen 1 (middle) of the adherent cells as the reference data in accordance with at least one example embodiment.

FIG. 14 is a graphical illustration demonstrating cell count data (oxygen) obtained by simulating the specimen 2 (low) using the specimen 1 (middle) of the adherent cells as the reference data in accordance with at least one example embodiment.

FIG. 15 is a graphical illustration demonstrating cell count data (carbon dioxide) obtained by simulating the specimen 2 (low) using the specimen 1 (middle) of the adherent cells as the reference data in accordance with at least one example embodiment.

FIG. 16 is a graphical illustration demonstrating cell count data (glucose) obtained by simulating the specimen 3 (high) using the specimen 1 (middle) of the adherent cells as the reference data in accordance with at least one example embodiment.

FIG. 17 is a graphical illustration demonstrating cell count data (lactic acid) obtained by simulating the specimen 3 (high) using the specimen 1 (middle) of the adherent cells as the reference data in accordance with at least one example embodiment.

FIG. 18 is a graphical illustration demonstrating cell count data (oxygen) obtained by simulating the specimen 3 (high) using the specimen 1 (middle) of the adherent cells as the reference data in accordance with at least one example embodiment.

FIG. 19 is a graphical illustration demonstrating cell count data (carbon dioxide) obtained by simulating the specimen 3 (high) using the specimen 1 (middle) of the adherent cells as the reference data in accordance with at least one example embodiment.

FIG. 20 is a table illustrating example culture conditions in accordance with at least one example embodiment.

FIG. 21 is a table illustrating culture conditions in accordance with at least one example embodiment.

FIG. 22 is a table illustrating culture condition in accordance with at least one example embodiment.

FIG. 23 is a graph illustration demonstrating cell count data (glucose) obtained by simulating the specimen 2 (low) using the specimen 1 (middle) of the floating cells as the reference data in accordance with at least one example embodiment.

FIG. 24 is a graphical illustration demonstrating cell count data (lactic acid) obtained by simulating the specimen 2 (low) using the specimen 1 (middle) of the floating cells as the reference data in accordance with at least one example embodiment.

FIG. 25 is a graphical illustration demonstrating cell count data (oxygen) obtained by simulating the specimen 2 (low) using the specimen 1 (middle) of the floating cells as the reference data in accordance with at least one example embodiment.

FIG. 26 is a graphical illustration demonstrating cell count data (carbon dioxide) obtained by simulating the specimen 2 (low) using the specimen 1 (middle) of the floating cells as the reference data in accordance with at least one example embodiment.

FIG. 27 is a graphical illustration demonstrating cell count data (glucose) obtained by simulating the specimen 3 (high) using the specimen 1 (middle) of the floating cells as the reference data in accordance with at least one example embodiment.

FIG. 28 is a graphical illustration demonstrating cell count data (lactic acid) obtained by simulating the specimen 3 (high) using the specimen 1 (middle) of the floating cells as the reference data in accordance with at least one example embodiment.

FIG. 29 is a graphical illustration demonstrating cell count data (oxygen) obtained by simulating the specimen 3 (high) using the specimen 1 (middle) of the floating cells as the reference data in accordance with at least one example embodiment.

FIG. 30 is a graphical illustration demonstrating cell count data (carbon dioxide) obtained by simulating the specimen 3 (high) using the specimen 1 (middle) of the floating cells as the reference data in accordance with at least one example embodiment.

### DETAILED DESCRIPTION

Before culturing cells, a user predicts a transition in the number of cells (metabolic state) with reference to past data and creates a culture plan in accordance with the prediction. For example, the user may identify or create a culture period in which a desired number of cells can be recovered and may proceed to culture cells in a cell culture apparatus during the culture period.

When cells are actually cultured, however, the transition of the change in the number of cells might deviate from the prediction. When the transition of an actual change in the number of cells falls below the predicted transition, a desired number of cells is unable to be recovered in the created culture period. Methods for predicting the transition of the change in the number of cells using data of actual change in the number of cells during the cell culture are provided.

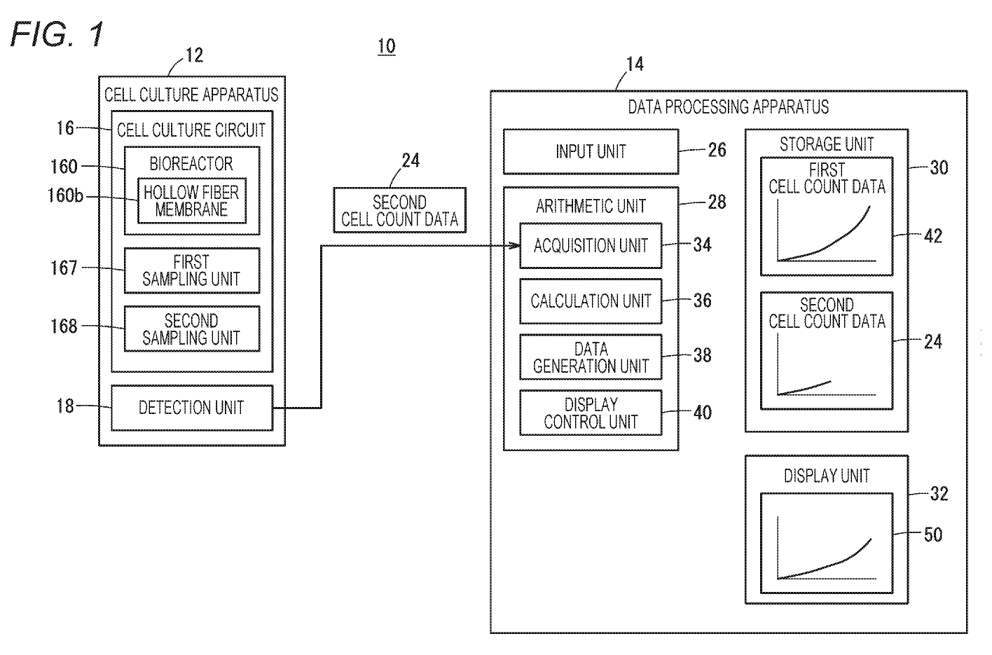

FIG. 1 is a block diagram of a cell culture system 10. The cell culture system 10 may include a cell culture apparatus 12 and a data processing apparatus 14. The data processing apparatus 14 may be configured to predict the transition of the change in the number of cells and to display the transition of the number of cells according to the prediction. The cells used in the cell culture system 10 may be mammalian cells or cells derived from a mammal. The cells used in the cell culture system 10 may be adherent cells or floating cells. Examples of the cells include, for example, human embryonic kidney cells (HEK 293 cells), embryonic stem cells (ES cells), induced pluripotent stem cells (iPS cells), mesenchymal stem cells, fibroblast cells, endothelial cells, neural stem cells, the like, or any combination thereof. Examples of the floating cells include Jurkat cells (human cellular leukemia-derived cells), T cells, regulatory T cells, tumor-infiltrating lymphocytes, CAR-T cells, CD34 positive cells, the like, or any combination thereof.

For cell culture, a basal medium and a complete medium may be used. The basal medium may include, for example, a minimum essential medium (MEM). The complete medium may include, for example, a basal medium (e.g., minimum essential medium (MEM)) including a nutrient component, like a protein. The protein may include albumin, growth factors, cytokines, the like, or any combination thereof . For example, in at least one example embodiment, bovine serum that includes albumin, growth factors, the like, or any combination thereof may be added to minimum essential medium (MEM) to form the basal medium.

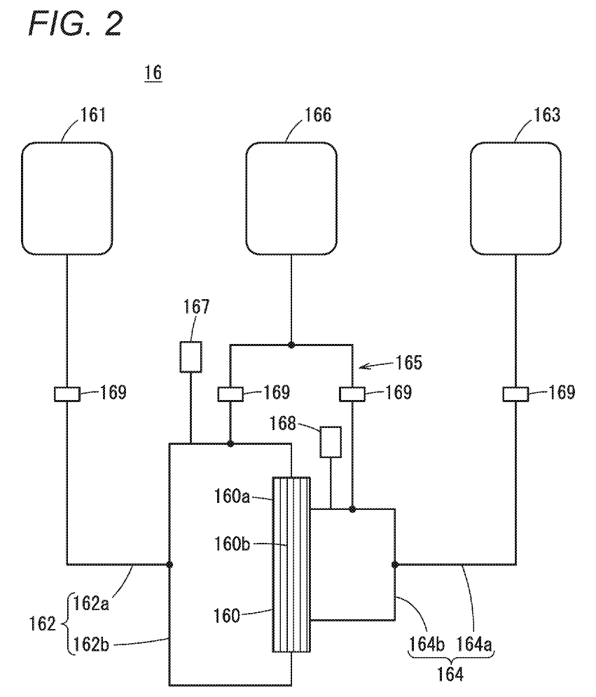

The cell culture apparatus 12 may include a cell culture circuit 16 and a detection unit 18. As illustrated in FIG. 2, the cell culture circuit 16 may include a bioreactor 160, a first supply unit 161, a first circuit 162, a second supply unit 163, a second circuit 164, a third circuit 165, a waste liquid storage unit 166, a first sampling unit 167, and a second sampling unit 168. Although not illustrated, it should be appreciated that, in various example embodiments, the first sampling unit 167 and the second sampling unit 168 may each include various sensors that are configured to detect concentrations of glucose, lactic acid, protein, the like, or any combination thereof in the cell culture. The cell culture circuit 16 may include various gas sensors that are configured to detect concentrations of gases (such as O₂ and CO₂) in the liquid. Although not illustrated, it should be appreciated that, in various example embodiments, the cell culture circuit 16 may include a plurality of pumps that are configured to apply a flow force to the liquid. The cell culture circuit 16 may include a plurality of valves 169 that can open and close the flow path.

The bioreactor 160 may include a housing 160a and a plurality of hollow fiber membranes 160b. The housing 160a may be configured to store a plurality of hollow fiber membranes 160b. Each hollow fiber membrane 160b may have a cylindrical shape. For example, each hollow fiber membrane 160b may include a pore (inner pore) that extends from one end to the other end. The bioreactor 160 may include a flow path defined by an inner peripheral surface of the hollow fiber membrane 160b and a flow path defined by an inner wall surface of the housing 160a and an outer peripheral surface of the hollow fiber membrane 160b. Inside the housing 160a, a fluid may flow from the inside to the outside of the hollow fiber membrane 160b and from the outside to the inside of the hollow fiber membrane 160b.

The first supply unit 161 may include a bag storing a liquid, such as a cell fluid, the complete medium, the basal medium, a cleaning liquid, or any combination thereof. The first circuit 162 may include a first supply flow path 162a and a first circulation flow path 162b. The first supply flow path 162a may connect the first supply unit 161 and the first circulation flow path 162b. The first circulation flow path 162b may include a flow path defined by the inner peripheral surface of each hollow fiber membrane 160b of the bioreactor 160. The liquid is supplied from the first supply unit 161 to the first circulation flow path 162b via the first supply flow path 162a may circulate in the first circulation flow path 162b. The first sampling unit 167 may be configured to sample the culture medium from the first circulation flow path 162b.

The second supply unit 163 may include a bag storing the liquid, such as the basal medium, the cleaning liquid, or a combination of the basal medium and the cleaning liquid. The second circuit 164 may include a second supply flow path 164a and a second circulation flow path 164b. The second supply flow path 164a may connect the second supply unit 163 and the second circulation flow path 164b. The second circulation flow path 164b may include the flow path defined by the inner wall surface of the housing 160a of the bioreactor 160 and the outer peripheral surface of the hollow fiber membrane 160b. The liquid supplied from the second supply unit 163 to the second circulation flow path 164b via the second supply flow path 164a may circulate in the second circulation flow path 164b. The second sampling unit 168 may be configured to sample the culture medium from the second circulation flow path 164b.

The third circuit 165 may connect the first circulation flow path 162b and the waste liquid storage unit 166 and also the second circulation flow path 164b and the waste liquid storage unit 166. The waste liquid storage unit 166 may include a container for storing a waste liquid. The waste liquid discharged from the first circulation flow path 162b and the second circulation flow path 164b may be recovered in the waste liquid storage unit 166.

A first valve the plurality of valves 169 may be disposed in the first supply flow path 162a. A second valve of the plurality of valves 169 may be disposed in the second supply flow path 164a. A third valve of the plurality of valves 169 may be disposed in the flow path of the third circuit 165 connecting the first circulation flow path 162b and the waste liquid storage unit 166. A fourth valve of the plurality of valves 169 may be disposed in the flow path of the third circuit 165 connecting the second circulation flow path 164b and the waste liquid storage unit 166.

With renewed reference to FIG. 1, the detection unit 18 may be configured to detect a second cell count data 24 from the culture medium sampled by at least one of the first sampling unit 167 and the second sampling unit 168. The detection unit 18 may be configured to transmit the second cell count data 24 to the data processing apparatus 14, for example, at a predetermined or selected time. The second cell count data 24 indicates a change (increase or decrease) in the number of cells in the instance of a second culture condition. The second cell count data 24 may include data obtained by combining metabolic rates of all cells present in the bioreactor 160. Cell count data (including the second cell count data 24 and also a first cell count data 42 and a third cell count data 44 later described) may include data that indicates concentrations of glucose, lactic acid, O₂, CO₂ the like, or any combination thereof in the culture medium. The second culture condition may include information regarding a substance contained in the culture medium, information regarding a gas supplied to the culture medium, information regarding the cell culture apparatus 12 (e.g., capacity information, operation information, culture form, the like, or any combination thereof), the number of culture days, seeding times, various temperatures, the like, or any combination thereof.

The data processing apparatus 14 may include an input unit 26, an arithmetic unit 28, a storage unit 30, and a display unit 32. The data processing apparatus 14 may also include, for example, a computer (e.g., personal computer, tablet terminal, smartphone, the like, or any combination thereof). In at least one example embodiment, the arithmetic unit 28 and the storage unit 30 may be provided in a server. In at least one example embodiment, the display unit 32 may be provided in a terminal device.

The input unit 26 may include a man-machine interface operated by a user. For example, the input unit 26 may include a keyboard, a touch panel, a mouse, the like, or any combination thereof. The input unit 26 may be configured to transmit the information input by the user to the arithmetic unit 28.

In at least one example embodiment, the arithmetic unit 28 may be configured by a processor or processing circuit. The processor may include, for example, a central processing unit (CPU), a graphics processing unit (GPU), or a combination of a central processing unit (CPU) and a graphics processing unit (GPU)..

The arithmetic unit 28 may include an acquisition unit 34, a calculation unit 36, a data generation unit 38 (prediction data generation unit), and a display control unit 40. Each of the acquisition unit 34, the calculation unit 36, the data generation unit 38, and the display control unit 40 may be configured to be implemented by executing a program stored in the storage unit 30 by the arithmetic unit 28.

At least a part of the acquisition unit 34, the calculation unit 36, the data generation unit 38, and the display control unit 40 may be implemented by an integrated circuit such as an application specific integrated circuit (ASIC) or a field-programmable gate array (FPGA). At least a part of the acquisition unit 34, the calculation unit 36, the data generation unit 38, and the display control unit 40 may be configured by an electronic circuit. The electronic circuit may include a discrete device.

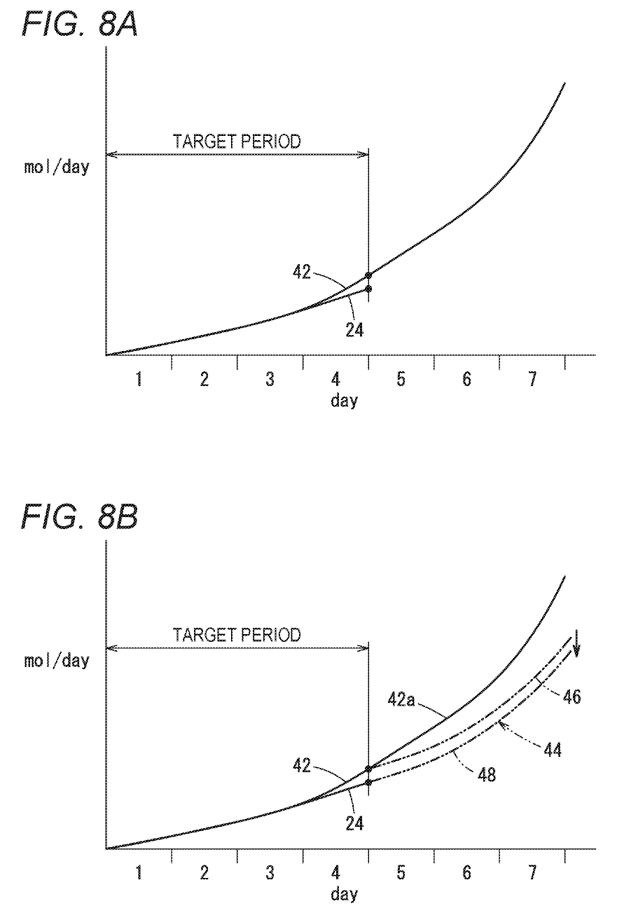

The acquisition unit 34 may be configured to acquire data from a device outside the arithmetic unit 28. For example, the acquisition unit 34 may be configured to acquire data from the cell culture apparatus 12, input unit 26, storage unit 30, external storage device, the like, or any combination thereof. The calculation unit 36 may be configured to calculate a first change rate, a second change rate, and a rate ratio, which is later described. The data generation unit 38 may be configured to generate correction data 46 and prediction data 48 (see, e.g., FIG. 8B) on the basis of the first cell count data 42 and the rate ratio. The data generation unit 38 may also be configured to generate the third cell count data 44 using the second cell count data 24 and the prediction data 48. The display control unit 40 may be configured to control the display of the display unit 32.

Although not illustrated, it should be appreciated that, in various example embodiments, the storage unit 30 may include a volatile memory and a non-volatile memory. The volatile memory may include a random access memory (RAM) and the like. The volatile memory may be used as a working memory of the processor and may be configured to temporarily store data and the like necessary for processing or arithmetic operation. The non-volatile memory may include a read only memory (ROM), a flash memory, or a combination thereof. The non-volatile memory may be used as a memory for storage and may be configured to store a program, a table, a map, the like, or any combination thereof. In at least one example embodiment, at least a part of the storage unit 30 may be provided in the processor, the integrated circuit, and the like as described above.

The storage unit 30 may be configured to store the first cell count data 42 before the cell culture apparatus 12 cultures the cells. The first cell count data 42 may include data indicating a change (increase or decrease) in the number of cells under a first culture condition. The first cell count data 42 may include data obtained by combining the metabolic rates of all cells present in the bioreactor 160. The first culture condition may include, for example, information regarding a substance contained in the culture medium, information regarding a gas supplied to the culture medium, information regarding the cell culture apparatus 12 (capacity information, operation information, culture form, the like, or any combination thereof), the number of culture days, seeding times, various temperatures, the like, or any combination thereof.

The first cell count data 42 may include data obtained by previously performed cell cultures. The first cell count data 42 may include, additionally or alternatively, data obtained by a simulation of cell culture. In at least one example embodiment, the first cell count data 42 may be stored in the external storage device and not the storage unit 30.

The display unit 32 may include a display. The display unit 32 may be configured to display various display objects (e.g.,, transition data 50) as controlled by the display control unit 40.

In the cell culture circuit 16, a controller may be configured to control a plurality of pumps, a plurality of valves 169, or a combination of pumps and valves 169 to change a flow path through which the culture medium flows and a flow rate of the culture medium, allowing the cell culture circuit 16 to implement a plurality of culture forms.

The first culture form and the second culture form may be primarily executed from an initial stage to a middle stage of a cell culture process. The third culture form and the fourth culture form may be primarily executed from the middle stage to a later stage of the cell culture process. The fifth culture form may be appropriately executed during the culture period in order to collect cells.

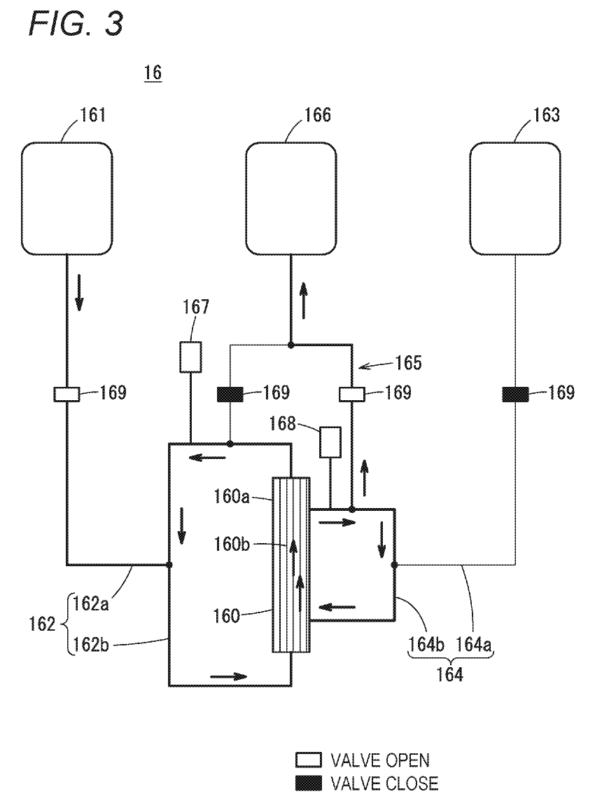

FIG. 3 is a diagram illustrating an operation of the cell culture apparatus 12 at the time of cell culture in the first culture form. The first culture form may include: (a) supplying the complete medium from the first supply unit 161 to the first circuit 162 (first circulation flow path 162b); (b) not supplying the basal medium from the second supply unit 163 to the second circuit 164 (second circulation flow path 164b); and (c) discarding a part of the culture medium from the second circulation flow path 164b to the waste liquid storage unit 166 via the third circuit 165.

The first culture form may be primarily executed in the cell culture process but may also be executed in a cell collection process in certain variations. When the first culture form is executed in the cell collection process, the basal medium may be supplied from both ends of the hollow fiber membrane 160b. As a result, the cells present in the first circulation flow path 162b may be collected in the hollow fiber membrane 160b.

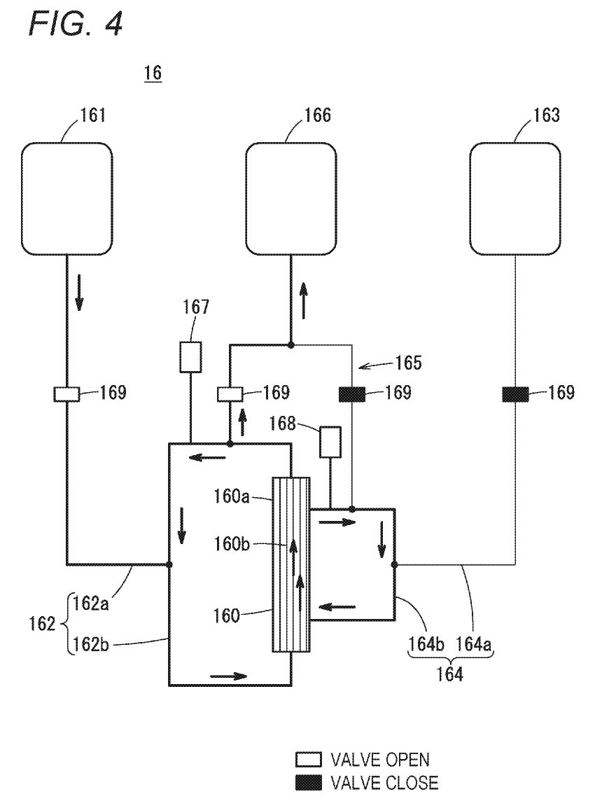

FIG. 4 is a diagram illustrating an operation of the cell culture apparatus 12 at the time of cell culture in the second culture form. The second culture form may include:(a) supplying the complete medium from the first supply unit 161 to the first circuit 162 (first circulation flow path 162b); (b) not supplying he basal medium from the second supply unit 163 to the second circuit 164 (second circulation flow path 164b); and(c) discarding a part of the culture medium from the first circulation flow path 162b to the waste liquid storage unit 166 via the third circuit 165.

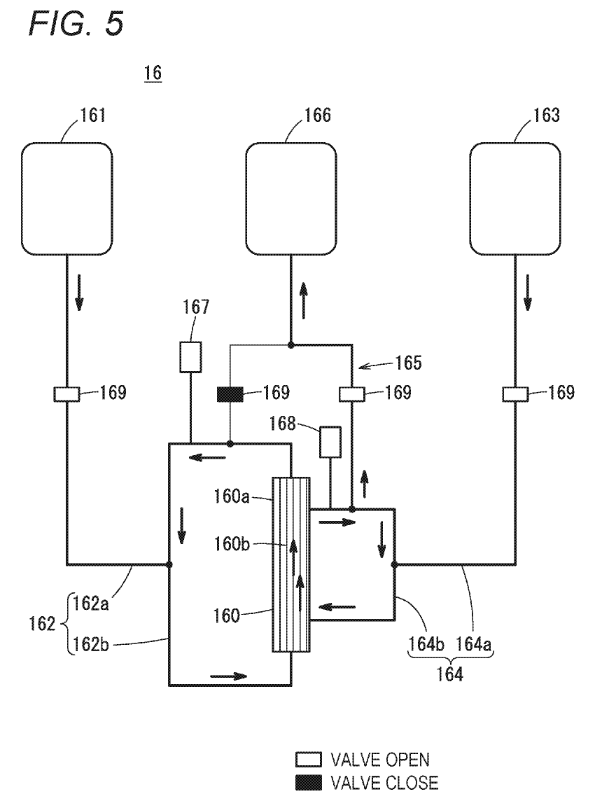

FIG. 5 is a diagram illustrating an operation of the cell culture apparatus 12 at the time of cell culture in the third culture form. The third culture form may include:(a) supplying the complete medium from the first supply unit 161 to the first circuit 162 (first circulation flow path 162b); (b) supplying the basal medium from the second supply unit 163 to the second circuit 164 (second circulation flow path 164b); and (c) discarding a part of the culture medium from the second circulation flow path 164b to the waste liquid storage unit 166 via the third circuit 165.

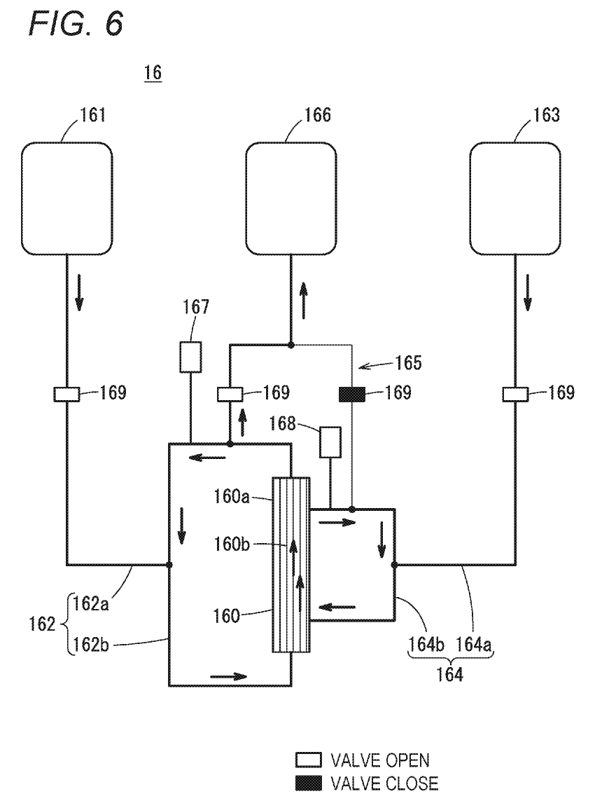

FIG. 6 is a diagram illustrating an operation of the cell culture apparatus 12 at the time of cell culture in the fourth culture form. The fourth culture form may include: (a) supplying the complete medium from the first supply unit 161 to the first circuit 162 (first circulation flow path 162b); (b) supplying the basal medium from the second supply unit 163 to the second circuit 164 (second circulation flow path 164b); and (c) discarding a part of the culture medium from the first circulation flow path 162b to the waste liquid storage unit 166 via the third circuit 165.

The fifth culture form may include (a) supplying the basal medium from the first supply unit 161 to the first circuit 162 (first circulation flow path 162b); (b) not supplying the basal medium from the second supply unit 163 to the second circuit 164 (second circulation flow path 164b); and(c) discarding a part of the culture medium from the second circulation flow path 164b to the waste liquid storage unit 166 via the third circuit 165. That is, an Operation of the cell culture apparatus 12 at the time of cell culture in the fifth culture form may be the same as the operation of the cell culture apparatus 12 at the time of cell culture in the first culture form (FIG. 3) except for step(a).

During the fifth culture form, the basal medium may be supplied from both ends of the hollow fiber membrane 160b as in the first culture form. As a result, the cells present in the first circulation flow path 162b may be collected in the hollow fiber membrane 160b. The fifth culture form may be defined as the cell collection process.

The sixth culture form may include(a) supplying the basal medium from the first supply unit 161 to the first circuit 162 (first circulation flow path 162b);(b) not supplying the basal medium from the second supply unit 163 to the second circuit 164 (second circulation flow path 164b); and (c) discarding a part of the culture medium is from the first circulation flow path 162b to the waste liquid storage unit 166 via the third circuit 165.. That is, an operation of the cell culture apparatus 12 at the time of cell culture in the sixth culture form may be the same as the operation of the cell culture apparatus 12 at the time of cell culture in the second culture form (FIG. 4) except for step (a).

The seventh culture form may include(a)supplying the basal medium from the first supply unit 161 to the first circuit 162 (first circulation flow path 162b);(b) supplying the basal medium from the second supply unit 163 to the second circuit 164 (second circulation flow path 164b); and (c) discarding a part of the culture medium is from the second circulation flow path 164b to the waste liquid storage unit 166 via the third circuit 165.. An operation of the cell culture apparatus 12 at the time of cell culture in the seventh culture form may be the same as the operation of the cell culture apparatus 12 at the time of cell culture in the third culture form (FIG. 5) except for step (a).

The eighth culture form may include (a) supplying the basal medium from the first supply unit 161 to the first circuit 162 (first circulation flow path 162b);(b) supplying the basal medium from the second supply unit 163 to the second circuit 164 (second circulation flow path 164b); and (c) discarding a part of the culture medium from the first circulation flow path 162b to the waste liquid storage unit 166 via the third circuit 165.. That is, an operation of the cell culture apparatus 12 at the time of cell culture in the eighth culture form may be the same as the operation of the cell culture apparatus 12 at the time of cell culture in the fourth culture form (FIG. 6) except for step (a).

The first culture form, the third culture form, the fifth culture form, and the seventh culture form may be executed in a culture process of floating cells. The second culture form, the fourth culture form, the sixth culture form, and the eighth culture form may be executed in a culture process of adherent cells. In culture processing of floating cells, the adherent cells, of a combination thereof one culture form among the detailed culture forms may be executed or a combination of one or more of the detailed culture forms may be executed.

The culture forms may be different in a supplied culture medium, a method of supplying the culture medium, a method of discarding the culture medium, the like, or any combination thereof. In a case of executing the generation of the third cell count data 44 (FIG. 8B) in any period, the data generation unit 38 may be configured to execute a simulation obtained by combining a plurality of culture forms.

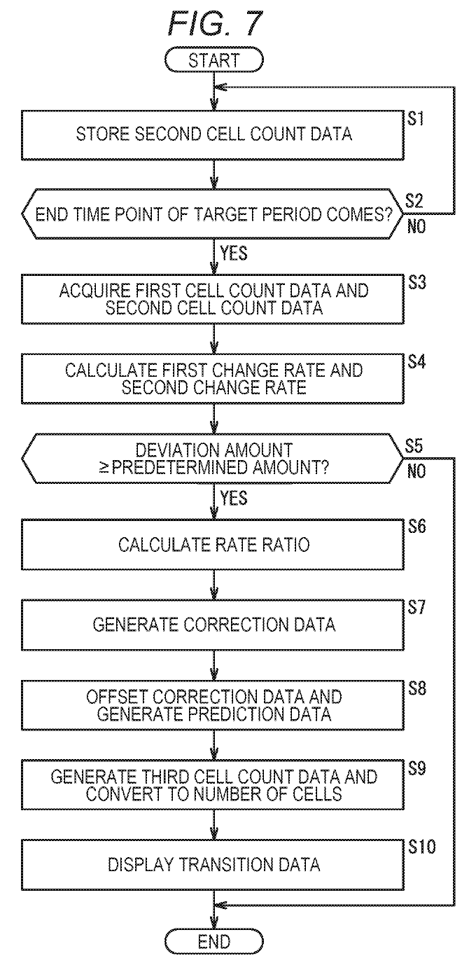

FIG. 7 is a flowchart of data display processing. The user may cause the cell culture apparatus 12 to execute the cell culture and the data processing apparatus 14 to execute the data display processing.

The user may communicate with the input unit 26 to designate a target period (e.g., a first predetermined or selected period and a second predetermined or selected period) before allowing the data processing apparatus 14 to execute the data display processing. The target period may be a period necessary for calculating the first change rate and the second change rate. The target period may be a period selected by the user out of the cell culture period. For example, when the cell culture period lasts for seven days, the user may input “first day” as a start time point of the target period and input “fourth day” as an end time point of the target period. Hereinafter, the target period will be described as “from first day to fourth day”. The user may also input other periods. The first predetermined or selected period that is the target period of the first cell count data 42 and the second predetermined or selected period that is the target period of the second cell count data 24 may be the same duration. That is, the second predetermined or selected period may correspond to the first predetermined or selected period. After inputting the target period, the user may communicate with the input unit 26 to cause the data processing apparatus 14 to execute data display processing.

At step S1, the acquisition unit 34 may be configured to acquire the second cell count data 24 from the detection unit 18 of the cell culture apparatus 12. The storage unit 30 may be configured to sequentially stores the acquired second cell count data 24.

At step S2, the calculation unit 36 may be configured to determine whether the fourth day (the end point of the target period) comes. In a case where the fourth day comes (step S2: YES), the processing may proceed to step S3. In contrast, in a case where the fourth day does not come (step S2: NO), the processing may return to step S1.

When the processing proceeds from step S2 to step S3, the acquisition unit 34 may be configured to acquire the first cell count data 42 and the second cell count data 24 from the storage unit 30. The first cell count data 42 acquired here may include data indicating a change in the number of cells from the start of culture to the end of culture under the first culture condition. The second cell count data 24 acquired here may include data indicating a change in the number of cells from the start of culture to a present time under the culture condition (second culture condition) of the cell culture currently being performed.

At step S4, the calculation unit 36 may be configured to calculate the first change rate and the second change rate in the target period designated by the user. The change rate may be a ratio between a designated target period (from first day to fourth day) and a change amount of the cell count data during the target period. For example, the calculation unit 36 may be configured to calculate the first change rate by dividing the change amount of the first cell count data 42 in the target period (first predetermined period) of the first cell count data 42 by the target period. Similarly, the calculation unit 36 may be configured to calculate the second change rate by dividing the change amount of the second cell count data 24 in the target period (second predetermined period) of the second cell count data 24 by the target period.

At step S5, the calculation unit 36 may be configured to calculate a deviation amount of the second change rate from the first change rate. Furthermore, the calculation unit 36 may be configured to compare the deviation amount with a predetermined or selected amount. The predetermined or selected amount may be a threshold for determining whether to execute the processing at and after step S6. That is, the predetermined or selected amount may be a threshold for determining whether to display the transition data 50 on the display unit 32. The predetermined or selected amount may be stored in advance in the storage unit 30. In a case where the deviation amount is the predetermined amount or larger (step S5: YES), the processing may proceed to step S6. In this case, as illustrated in FIG. 8A, a deviation may already occur between the first cell count data 42 and the second cell count data 24 on the fourth day (the end point of the target period). In this case, there may be a high possibility that the transition of the second cell count data 24 on and after the fourth day significantly deviates from the transition of the first cell count data 42. That is, at the end of the cell culture period, the number of cells may be likely to be significantly less than the initially predicted number. In contrast, in a case where the deviation amount is less than the predetermined amount (step S5: NO), the data display processing may end. In this case, there may be a high possibility that the transition of the second cell count data 24 after the fourth day is not significantly deviated from the transition of the first cell count data 42. That is, at the end of the cell culture period, the number of cells is likely to approximate the initially predicted number. Therefore, it is not necessary to display the transition data 50 and the data display processing ends.

In a case where the processing proceeds from step S5 to step S6, processing of predicting the transition of the second cell count data 24 after the fourth day may be executed. For example, the calculation unit 36 may be configured to calculate the rate ratio. The rate ratio may be a ratio between the first change rate and the second change rate. For example, the calculation unit 36 may be configured to calculate the rate ratio by dividing the second change rate by the first change rate.

At step S7, the data generation unit 38 may be configured to generate the correction data 46 by correcting the first cell count data 42. As illustrated in FIG. 8B, for example, the data generation unit 38 may be configured to generates the correction data 46 on the basis of first partial data 42a of the first cell count data 42 and the rate ratio. The first partial data 42a may include partial data after the target period in the first cell count data 42. Here, the first partial data 42a may include data from fifth day to seventh day of the first cell count data 42. The data generation unit 38 may be configured to generate the correction data 46 by multiplying the first partial data 42a by the rate ratio.

At step S8, the data generation unit 38 may be configured to bring the correction data 46 close to the second cell count data 24. Specifically, the data generation unit 38 may be configured to offset the correction data 46 by a difference between the first cell count data 42 and the second cell count data 24 on the fourth day (the end point of the target period). As a result, the data generation unit 38 may be configured to generate the prediction data 48. As described above, the correction data 46 after offset may be referred to as the prediction data 48. The prediction data 48 may include data indicating a change in the number of cells after the present time under the second culture condition.

At step S9, the data generation unit 38 may be configured to generate the third cell count data 44 (FIG. 8B) obtained by combining the prediction data 48 with the second cell count data 24. Furthermore, the data generation unit 38 may be configured to convert the third cell count data 44 into the number of cells by a predetermined method.

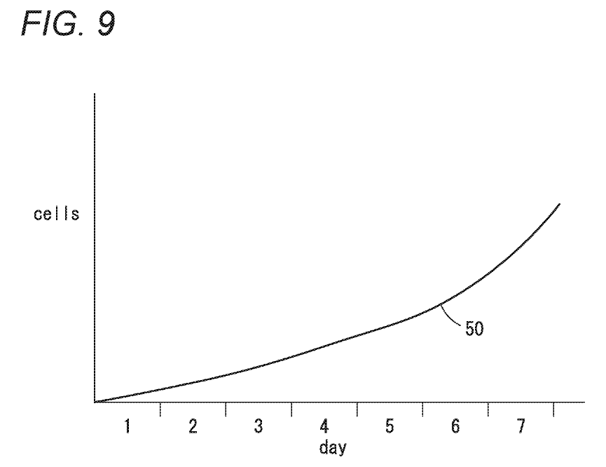

At step S10, the data generation unit 38 may be configured to perform fitting using the data of the number of cells after the conversion and to generate the transition data 50 indicating the transition of the number of cells. The display control unit 40 may be configured to perform control to display the transition data 50 on the display unit 32. Then, the display unit 32 may be configured to display the transition data 50 as illustrated in FIG. 9. The display control unit 40 may be configured to perform control to display the third cell count data 44 on the display unit 32 together with the transition data 50 and/or to perform control to display only the third cell count data 44 on the display unit 32.

There may be instances where pieces of cell count data is present before the cell culture. For example, when there is cell count data obtained in one or more prior runs of cell culture and/or when there is cell count data obtained from one or more simulations, the acquisition unit 34 may be configured to acquire the first cell count data 42 using the available cell count data. For example, the acquisition unit 34 may be configured to calculate an average of cell count data to obtain the first cell count data 42.

Although not illustrated, it should be appreciated that, in various example embodiments, the order of processing of multiplying the rate ratio at step S7 and offset processing at step S8 may be switched. That is, the data generation unit 38 may offset the first partial data 42a and use the first partial data 42a after the offset as the correction data. Furthermore, the data generation unit 38 may be configured to generate the prediction data 48 by multiplying the correction data by the rate ratio.

The display control unit 40 may be configured to allow the display unit 32 to display the transition data 50 and to display an allowable range of the number of cells. The storage unit 30 may be configured to store the allowable range of the number of cells in advance.

The data generation unit 38 may be configured to calculate a timing (date and time) at which the number of cells designated by the user can be recovered on the basis of the transition data 50. The display control unit 40 may be configured to allow the display unit 32 to display the timing at which the number of cells designated by the user can be recovered.

In a case where the acquisition unit 34 acquires the second cell count data 24, the data generation unit 38 may be configured to calculate the allowable range of the cell count data. For example, the data generation unit 38 may be configured to calculate values of ±5%×day with respect to the second cell count data 24. The value of -5%×day with respect to the second cell count data 24 may be a lower limit value of the allowable range. The value of +5%×day with respect to the second cell count data 24 may be an upper limit value of the allowable range. The display control unit 40 may be configured to allow the display unit 32 to display the allowable range of the cell count data calculated by the data generation unit 38.

In the instance of each embodiment, the generation of the prediction data 48 (i.e., data during culture that reflects the second cell count data 24) may make it possible to predict the transition of culture during an ongoing cell culture and may allow the user to adjust an initial culture plan as indicated during the ongoing cell culture.

In the instance of each embodiment, the user may be able to determine the culture period during which a desired number of cells can be recovered using a number of cells as displayed on the display unit 32.

In the instance of each embodiment, the prediction data 48 may be created only when a difference between the initial culture plan and an actual culture situation is different from a predetermined or selected threshold (e.g., is larger than the predetermined or selected threshold.

It should be appreciated that the present disclosure is not limited to the above description, that various configurations can be adopted without departing from the gist of the present disclosure.

Certain features of the current technology are further illustrated in the following non-limiting examples.

Third cell count data (e.g., third cell count data 44) was generated in accordance with various aspects of the present disclosure. The generated third cell count data was compared to a measurement result measured in the actual cell culture to evaluate accuracy of the third cell count data. As further detailed below, the generated third cell count data was found to be close to the actual measurement result indicating the applicability of the third cell count data.

A Quantum cell expansion system (manufactured by Terumo BCT, Inc.) or a Quantum Flex^{TM} cell expansion system (manufactured by Terumo BCT, Inc.) was used as the cell culture apparatus (e.g., cell culture apparatus 12) in this example. The Quantum cell expansion system is hereafter referred to as a “first culture apparatus”. The Quantum Flex cell expansion system is hereafter referred to as a “second culture apparatus”. The bioreactor (e.g., bioreactor 160) size is different between the first culture apparatus and the second culture apparatus. The second culture apparatus has a bioreactor size that is 1/10 the size of the bioreactor of the first culture apparatus. In at least one of the first culture apparatus and the second culture apparatus, the size of the bioreactor may be able to be changed.

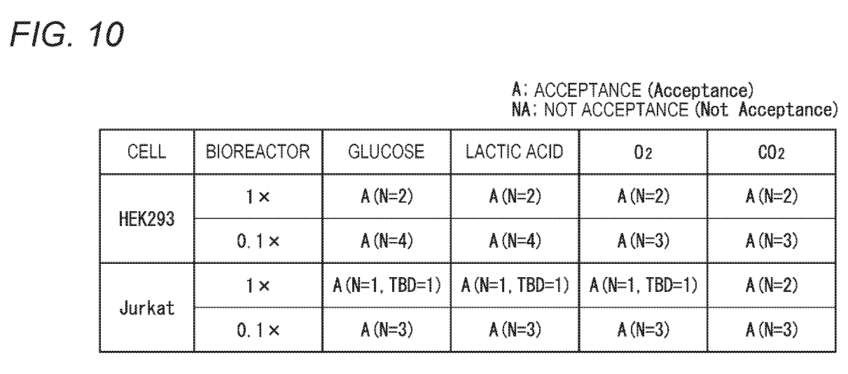

FIG. 10 is a table illustrating evaluation results. For example, in a column titled “cell”, cultured cells are entered. In a column titled “bioreactor”, the culture apparatus used is entered. In the column titled “bioreactor”, “1×” indicates that the first culture apparatus was used, and “0.1×” indicates that the second culture apparatus was used. In a column titled “glucose”, evaluation results are entered. In the column titled “glucose”, “A” indicates that the evaluation result is “acceptance”. In the present example, a simulation value falling within a range of ±5%×day with respect to a measured concentration was determined to indicate an “acceptance”. In this example, there were no example evaluated as “not acceptance” (NA). “N” in parentheses in the table indicates the number of pieces of evaluated processing (processing of generating the third cell count data). “TBD” in parentheses in the table indicates the number of specimens whose simulation value did not fall within the range of ±5%×day with respect to the measured concentration on the final day of culture. Similar to the “glucose” column, the evaluation results are also entered in respective columns for “lactic acid”, “O₂”, and “CO₂”.

As illustrated in FIG. 10, in the instance of the simulation including culturing Jurkat cells by the first culture apparatus (1×), a part of the evaluations of glucose and lactic acid was “TBD”. However, in the simulation period of seven days, both simulation values up to sixth day fell within the range of ±5%×day. Thus, in the instance of the simulation of culturing Jurkat cells by the first culture apparatus (1×), it was possible to comprehensively grasp the transition of metabolism.

For the condition resulting in “TBD”, the simulation value could likely be improved by performing the simulation again while changing the number of prediction days, increasing the frequency of data acquisition, increasing the number of reference data, the like, or any combination thereof.

### EXAMPLE 1

Three cell culture experiments were conducted under different culture conditions using the first culture apparatus to generate three specimens (specimen 1, specimen 2, and specimen 3) of the adherent cells. In each of the three cell culture experiments, cell culture was performed for seven days, and the cell count data was periodically measured. Processing of the generated third cell count data under the culture conditions of the specimens 2 and 3 was performed on the basis of the cell count data (e.g., first cell count data) obtained by the cell culture of the specimen 1. Further, the third cell count data was evaluated by comparing the third cell count data obtained under the culture condition of the specimen 2 with the cell count data obtained by the cell culture experiment of the specimen 2. Similarly, the third cell count data was evaluated by comparing the third cell count data obtained under the culture condition of the specimen 3 with the cell count data obtained by the cell culture experiment of the specimen 3.

The conditions under which the three cell culture experiments were carried out are further described below.

HEK293 cells were used as the adherent cells to be cultured.

Complete medium containing total protein as protein was used.

After priming the cell culture circuit (e.g., cell culture circuit 16) with PBS, the cell culture circuit 16 was precoated with cellular adhesion factors (fibronectin), and after the precoating, the bag filled with the complete medium was connected to the first supply flow path (e.g., first supply flow path 162a).

Gas conditioning was performed, and cells were seeded in the first circulation flow path (e.g., first circulation flow path 162b).

Cell culture was performed mainly in the second culture form.

During the cell culture period, the flow rate of the first circulation flow path (e.g., first circulation flow path 162b) was set to 20 mL/min, and the flow rate of the second circulation flow path (e.g., second circulation flow path 164b) was set to 300 mL/min.

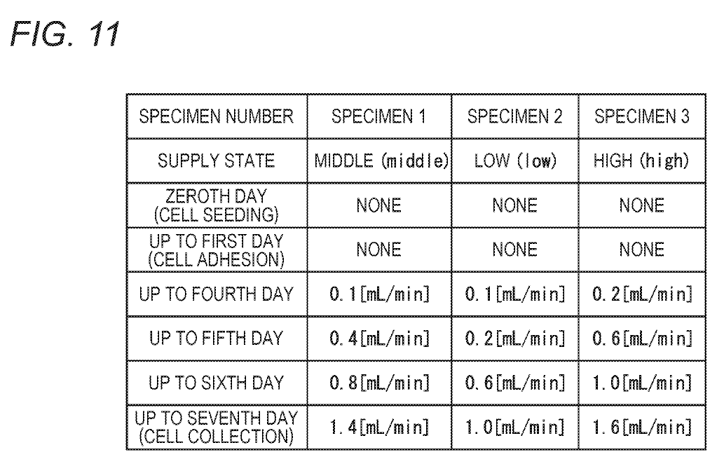

FIG. 11 is a table illustrating the culture condition of each of the three specimens. For example, FIG. 11 illustrates the flow rate of the complete medium supplied from the first supply unit (e.g., first supply unit 161) to the first circuit (e.g., first circuit 162) for each day.

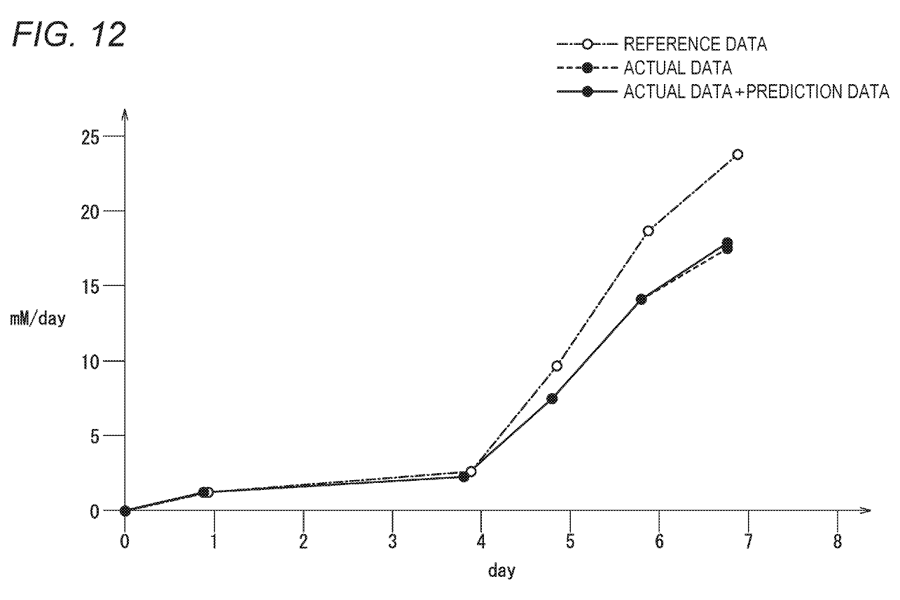

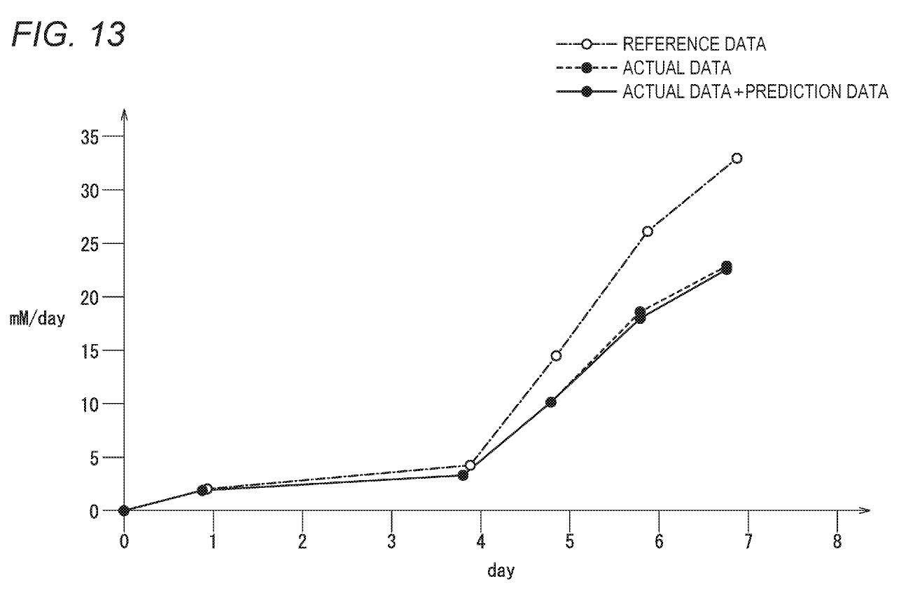

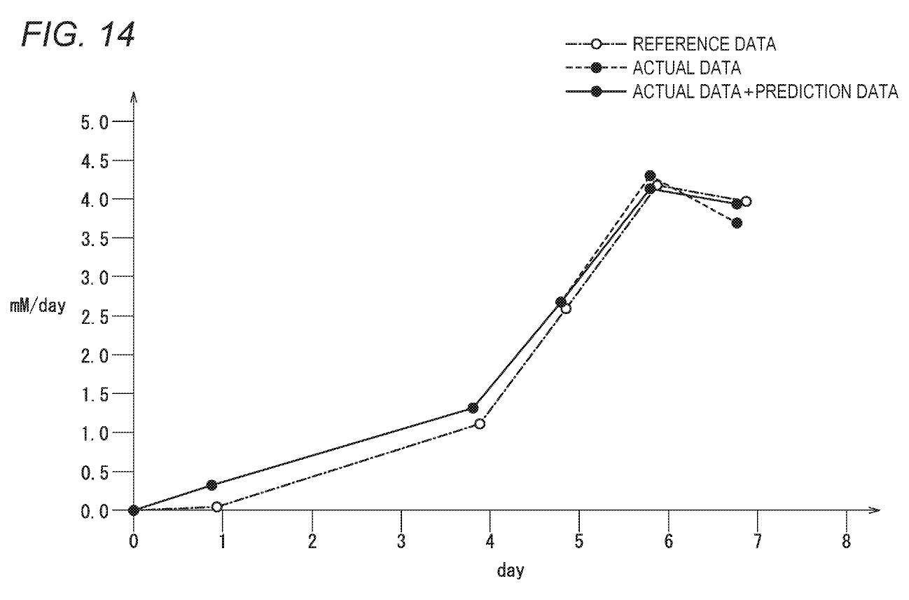

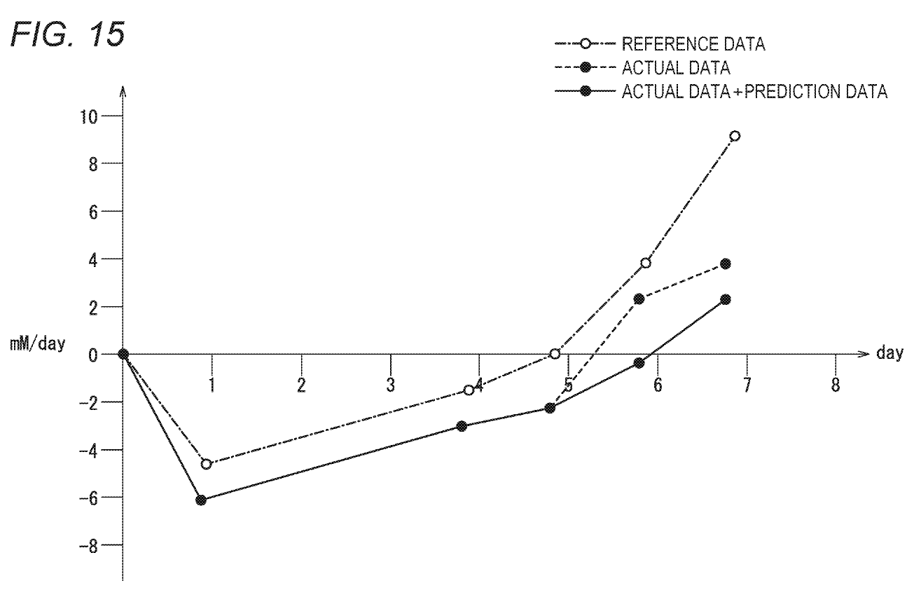

FIG. 12 is a graphical illustration demonstrating the cell count data (glucose) obtained by simulating the specimen 2 (low) using the specimen 1 (middle) as the reference data, where the x-axis represents day and the y-axis represents mM/day. FIG. 13 is a graphical illustration demonstrating the cell count data (lactic acid) obtained by simulating the specimen 2 (low) using the specimen 1 (middle) as the reference data, where the x-axis represents day and the y-axis represents mM/day. FIG. 14 is a graphical illustration demonstrating the cell count data (oxygen) obtained by simulating the specimen 2 (low) using the specimen 1 (middle) as the reference data, where the x-axis represents day and the y-axis represents mM/day. FIG. 15 is a graphical illustration demonstrating the cell count data (carbon dioxide) obtained by simulating the specimen 2 (low) using the specimen 1 (middle) as the reference data, where the x-axis represents day and the y-axis represents mM/day.

In each of FIGS. 12 to 15, the dash-dot line reflects the reference data. The reference data is the cell count data (i.e., first cell count data) obtained by the cell culture of the specimen 1. In each of FIGS. 12 to 15, the broken line reflects the actual data. The actual data is the cell count data obtained by the cell culture of the specimen 2. In each of FIGS. 12 to 15, the solid line reflects data (i.e., third cell count data) obtained by combining the actual data and the prediction data 48.

In each of FIGS. 12 to 15, the solid line substantially fell within ±5%×day of the broken line, indicating that the third cell count data was appropriately generated regarding the specimen 2 of the adherent cells.

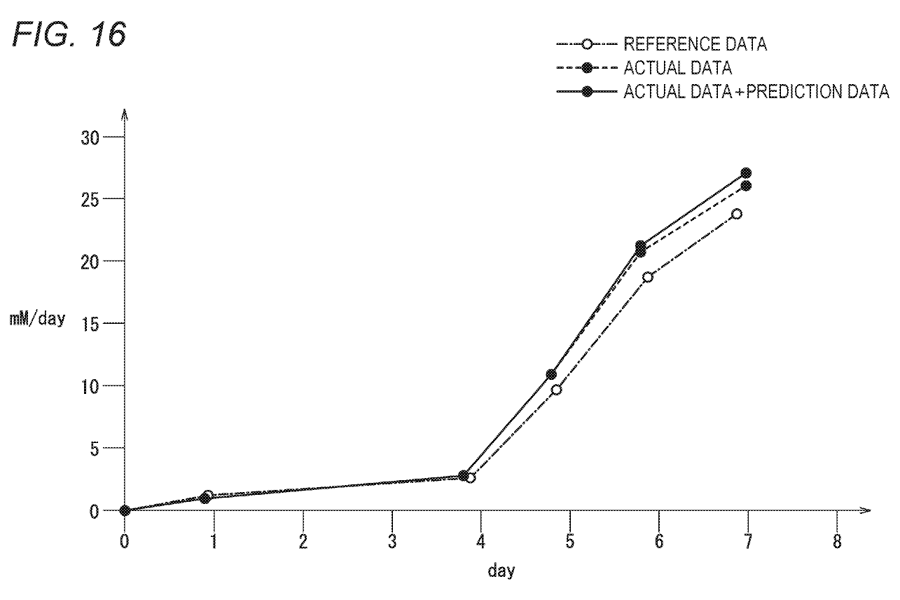

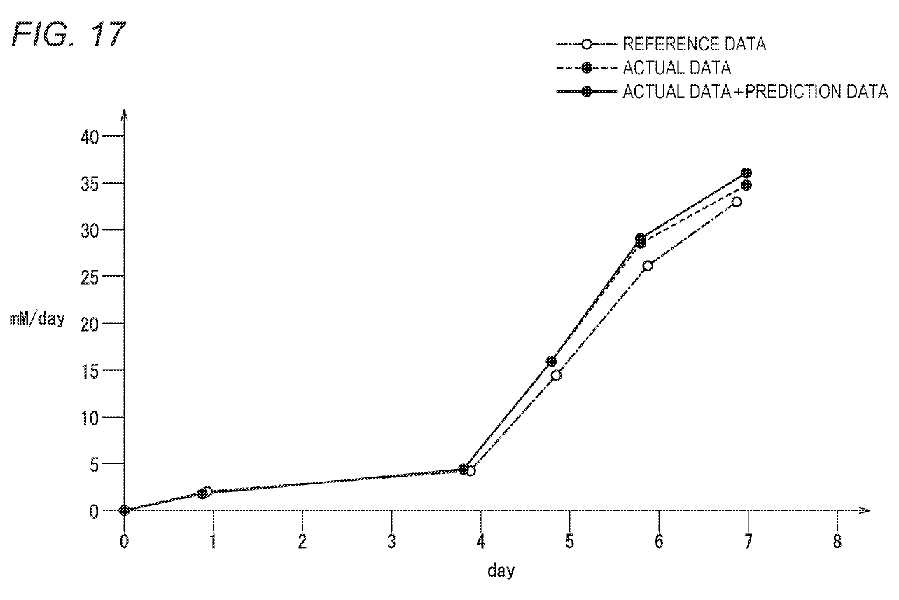

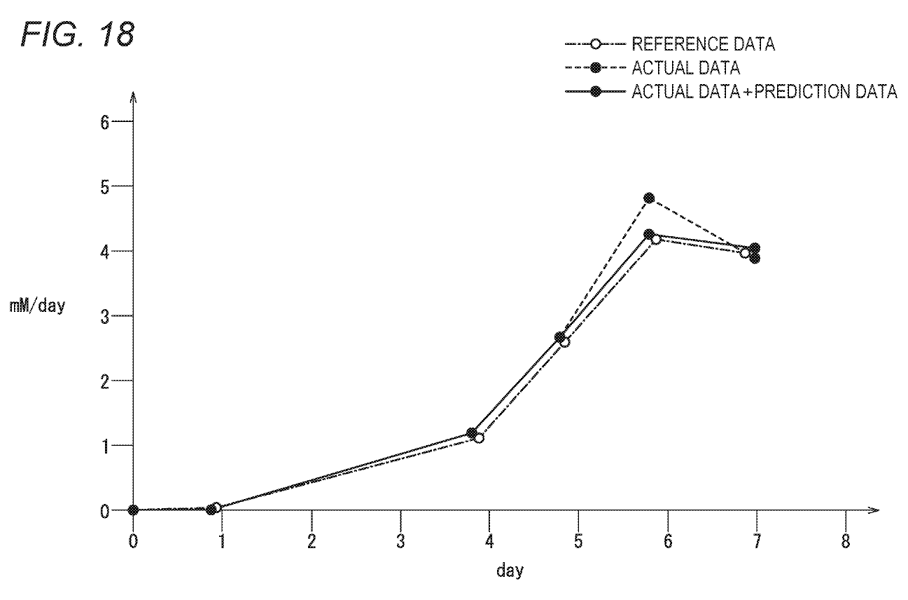

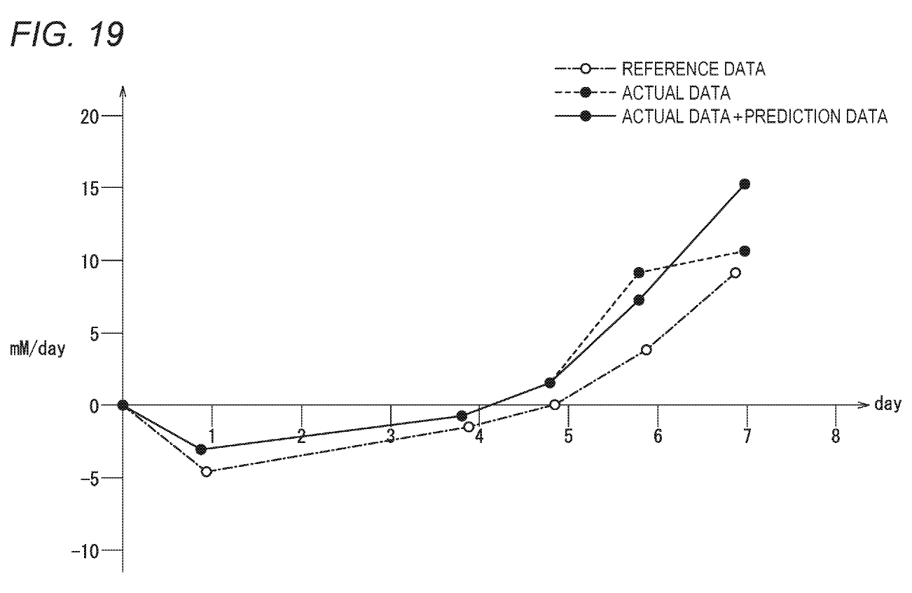

FIG. 16 is a graphical illustration demonstrating the cell count data (glucose) obtained by simulating the specimen 3 (high) using the specimen 1 (middle) of the adherent cells as the reference data, where the x-axis represents day and the y-axis represents mM/day. FIG. 17 is a graphical illustration demonstrating the cell count data (lactic acid) obtained by simulating the specimen 3 (high) using the specimen 1 (middle) of the adherent cells as the reference data, where the x-axis represents day and the y-axis represents mM/day. FIG. 18 is a graphical illustration demonstrating the cell count data (oxygen) obtained by simulating the specimen 3 (high) using the specimen 1 (middle) of the adherent cells as the reference data, where the x-axis represents day and the y-axis represents mM/day. FIG. 19 is a graphical illustration demonstrating the cell count data (carbon dioxide) obtained by simulating the specimen 3 (high) using the specimen 1 (middle) of the adherent cells as the reference data where the x-axis represents day and the y-axis represents mM/day.

In each of FIGS. 16 to 19, the dash-dot line reflects the reference data. The reference data is the cell count data (i.e., first cell count data) obtained by the cell culture of the specimen 1. In each of FIGS. 16 to 19, the broken line reflects actual data. The actual data is the cell count data obtained by the cell culture of the specimen 3. In each of FIGS. 16 to 19, the solid line reflects data (i.e., third cell count data) obtained by combining the actual data and the prediction data.

In each of FIGS. 16 to 19, the solid line substantially fell within ±5%×day of the broken line, indicating that the third cell count data was appropriately generated regarding the specimen 3 of the adherent cells.

### EXAMPLE 2

Three cell culture experiments were conducted under different culture conditions using the first culture apparatus to generate three specimens (specimen 1, specimen 2, and specimen 3) of the floating cells. In each of the three cell culture experiments, cell culture was performed for seven days, and the cell count data was periodically measured. Processing of the generated third cell count data under the culture conditions of the specimens 2 and 3 was performed on the basis of the cell count data (e.g., first cell count data) obtained by the cell culture of the specimen 1. Further, the third cell count data was evaluated by comparing the third cell count data obtained under the culture condition of the specimen 2 with the cell count data obtained by the cell culture experiment of the specimen 2. Similarly, the third cell count data was evaluated by comparing the third cell count data obtained under the culture condition of the specimen 3 with the cell count data obtained by the cell culture experiment of the specimen 3.

The conditions under which the three cell culture experiments were carried out are further described below.

Jurkat cells were used as the floating cells to be cultured.

Complete medium containing total protein as protein was used.

The cell culture circuit (e.g., cell culture circuit 16) was primed with PBS, and after the priming, the bag filled with the complete medium was connected to the first supply flow path (e.g., first supply flow path 162a).

Gas conditioning was performed, and cells were seeded in the first circulation flow path (e.g., first circulation flow path 162b).

In an initial stage of the cell culture period (seven days), a series of processes including the cell culture step, a cell dispersion step, and a cell collection step according to the first culture form were repeatedly performed. At the cell dispersion step, the supply and discard of the culture medium were stopped, and the culture medium was circulated in the first circulation flow path (e.g., first circulation flow path 162b). At the cell collection step, the cells diffused into the first circulation flow path (e.g., first circulation flow path 162b) at the cell dispersion step were collected inside the bioreactor (e.g., bioreactor 160). Here, the cell collection step was performed according to the fifth culture form.

In middle to later stages of the cell culture period (seven days), a series of processes including the cell culture step, a cell dispersion step, and a cell collection step according to the third culture form were repeatedly performed. Here, the cell collection step was performed according to the fifth culture form.

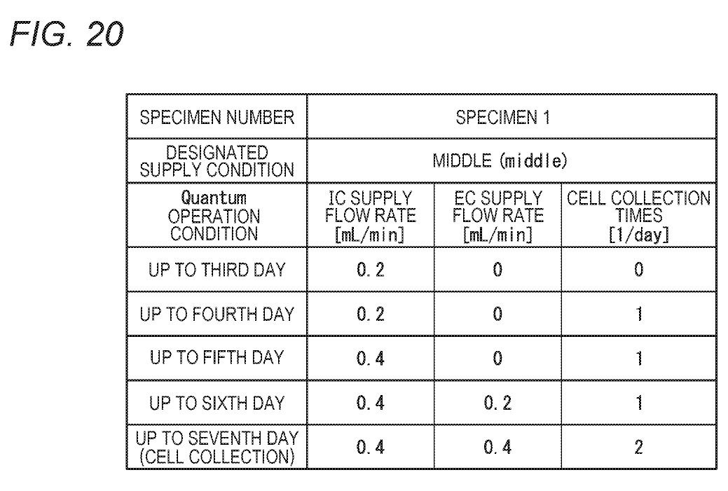

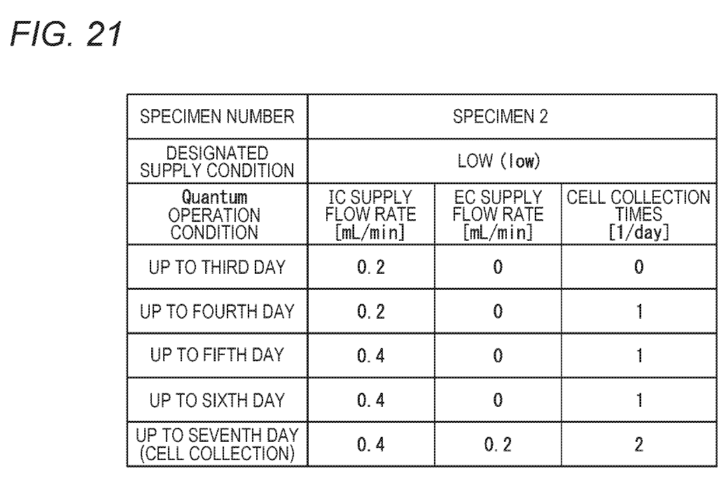

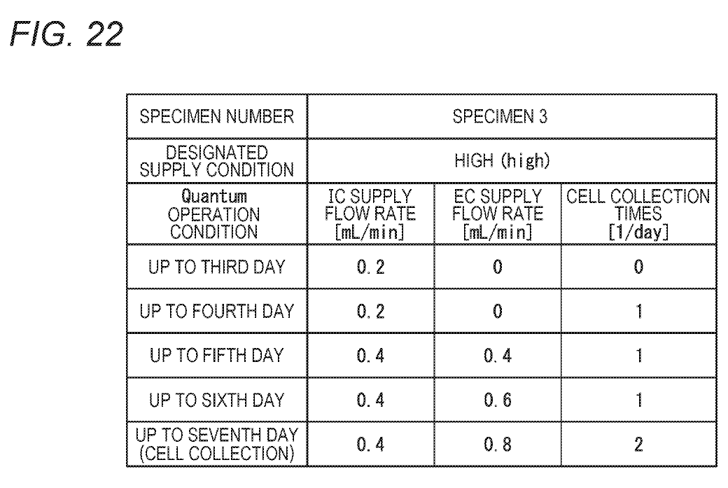

FIGS. 20 to 22 are tables illustrating the culture conditions. FIG. 20 illustrates the culture condition of the specimen 1. FIG. 21 illustrates the culture condition of the specimen 2. FIG. 22 illustrates the culture condition of the specimen 3. An “IC supply flow rate” illustrated in each table is the flow rate of the complete medium supplied from the first supply unit (e.g., first supply unit 161) to the first circulation flow path (e.g., first circulation flow path 162b) via the first supply flow path (e.g., first supply flow path 162a). An “EC supply flow rate” illustrated in each table is the flow rate of the basal medium supplied from the second supply unit (e.g., second supply unit 163) to the second circulation flow path (e.g., second circulation flow path 164b) via the second supply flow path (e.g., second supply flow path 164a).

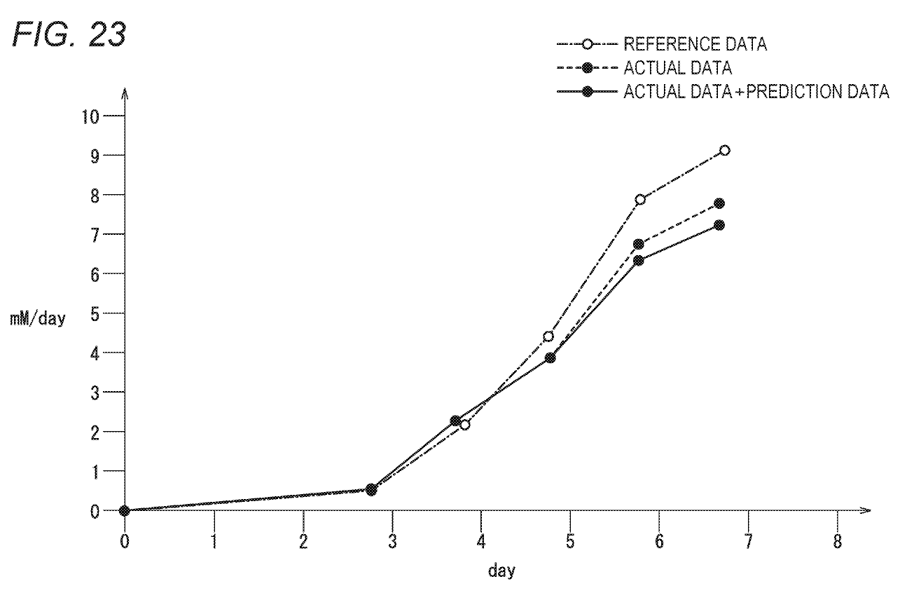

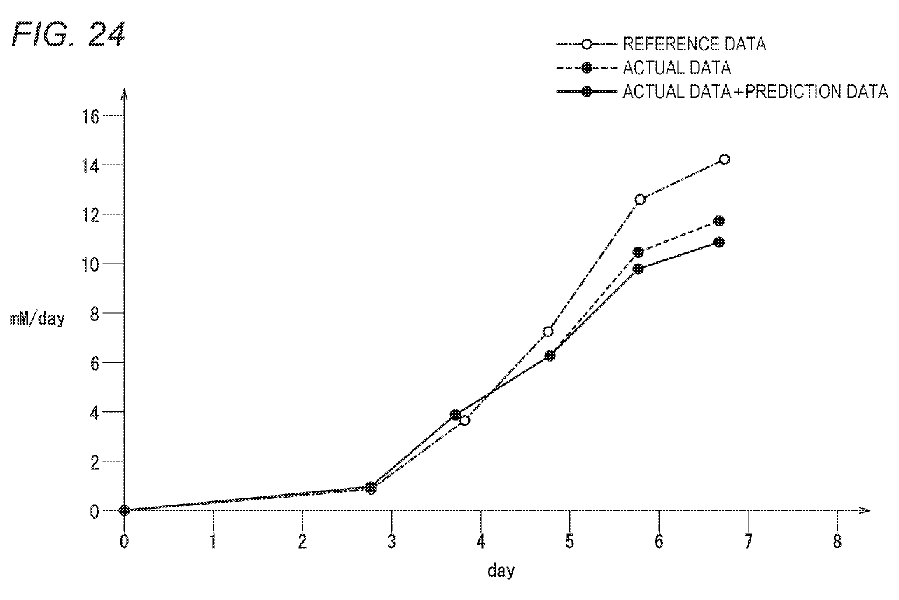

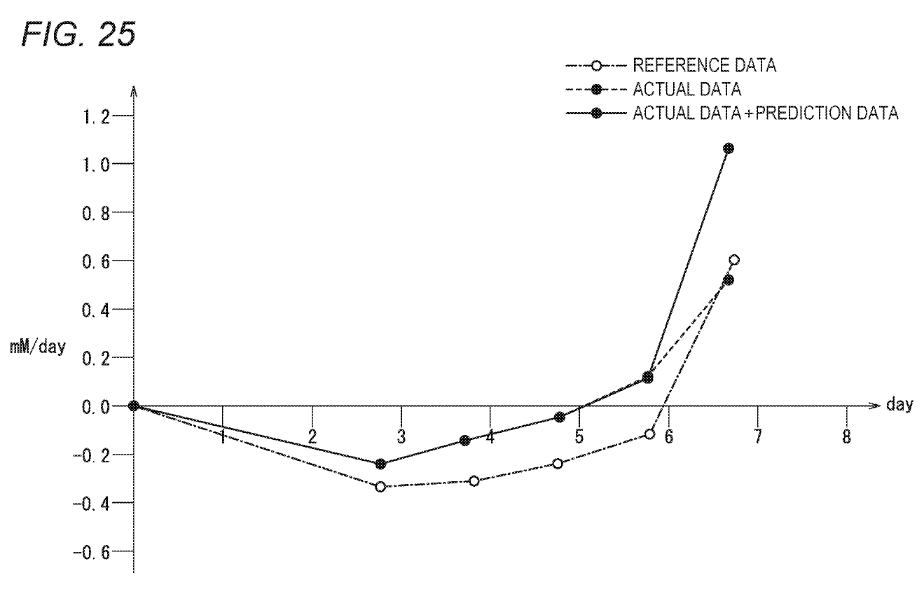

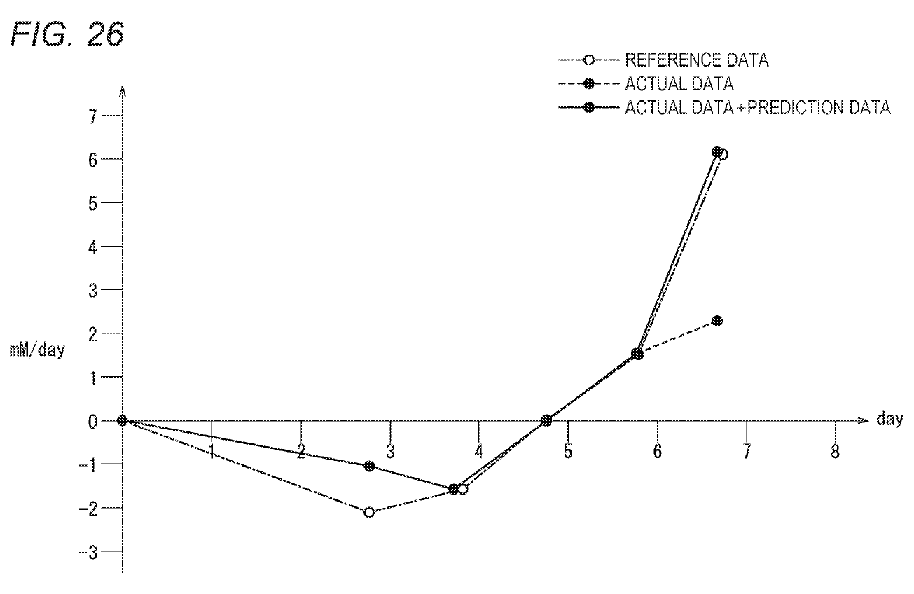

FIG. 23 is a graphical illustration demonstrating the cell count data (glucose) obtained by simulating the specimen 2 (low) using the specimen 1 (middle) of the floating cells as the reference data, where the x-axis represents day and the y-axis represents mM/day. FIG. 24 is a graphical illustration demonstrating the cell count data (lactic acid) obtained by simulating the specimen 2 (low) using the specimen 1 (middle) of the floating cells as the reference data, where the x-axis represents day and the y-axis represents mM/day. FIG. 25 is a graphical illustration demonstrating the cell count data (oxygen) obtained by simulating the specimen 2 (low) using the specimen 1 (middle) of the floating cells as the reference data, where the x-axis represents day and the y-axis represents mM/day. FIG. 26 is a graphical illustration demonstrating the cell count data (carbon dioxide) obtained by simulating the specimen 2 (low) using the specimen 1 (middle) of the floating cells as the reference data, where the x-axis represents day and the y-axis represents mM/day.

In each of FIGS. 23 to 26, the dash-dot line reflects the reference data. The reference data is the cell count data (e.g., first cell count data) obtained by the cell culture of the specimen 1. In each of FIGS. 23 to 26, the broken line reflects actual data. The actual data is the cell count data obtained by the cell culture of the specimen 2. In each of FIGS. 23 to 26, the solid line is data (e.g., third cell count data ) obtained by combining the actual data and the prediction data.

In each of FIGS. 23 to 26, the solid line substantially fell within ±5%×day of the broken line, indicating that the third cell count data 44 was appropriately generated regarding the specimen 2 of the floating cells.

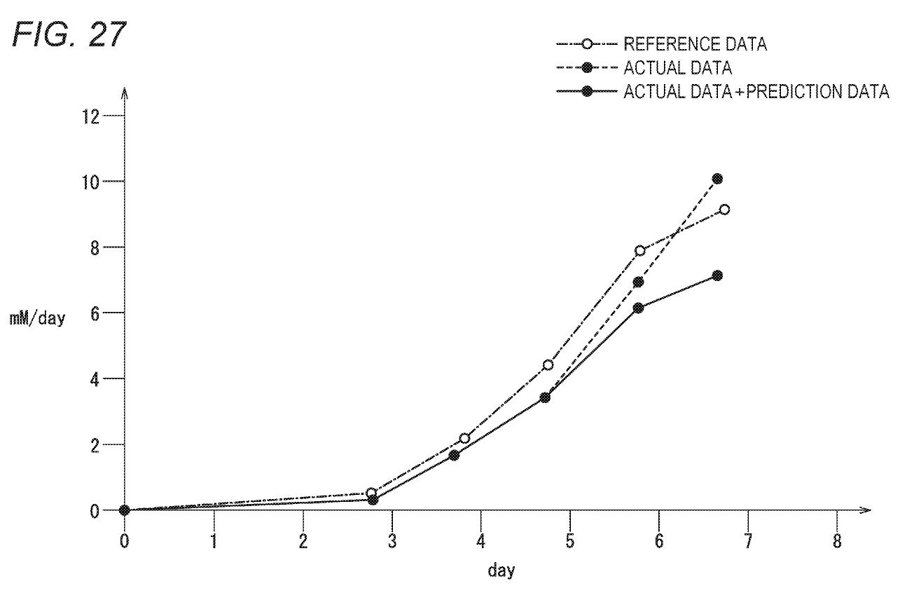

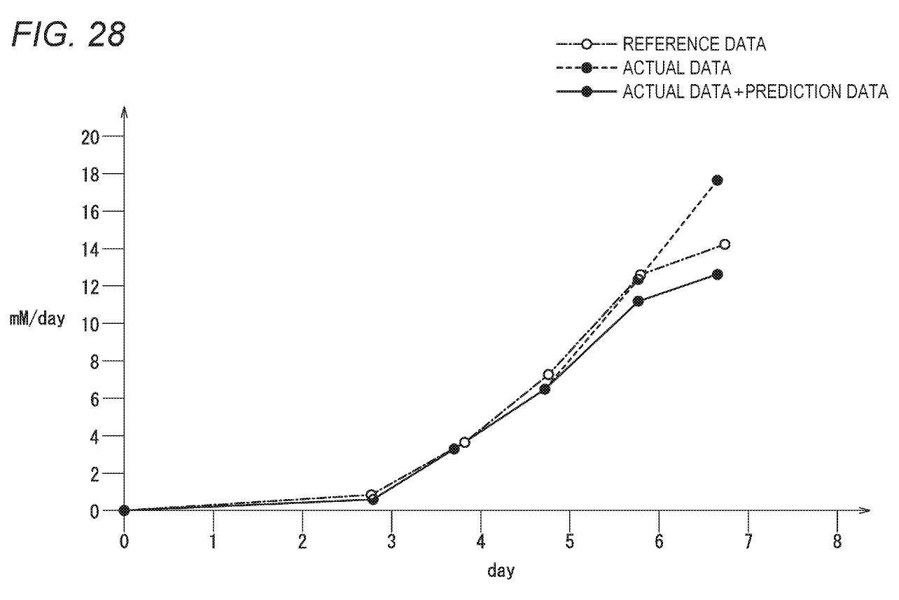

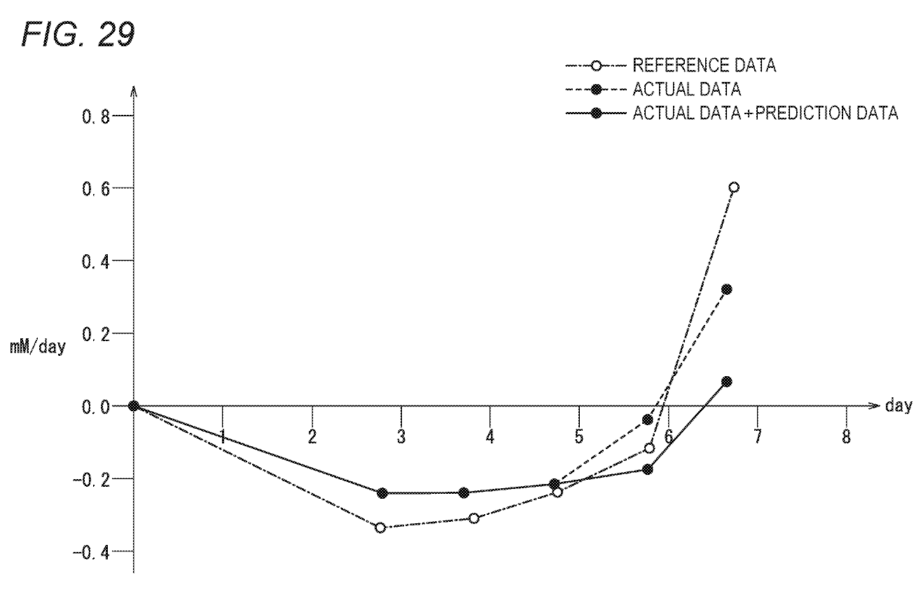

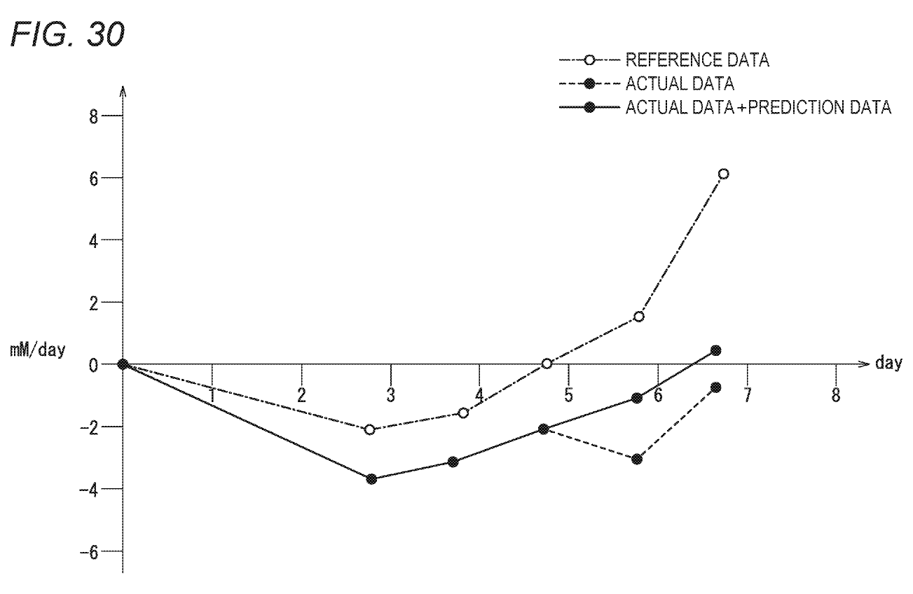

FIG. 27 is a graphical illustration demonstrating the cell count data (glucose) obtained by simulating the specimen 3 (high) using the specimen 1 (middle) of the floating cells as the reference data, where the x-axis represents day and the y-axis represents mM/day. FIG. 28 is a graphical illustration demonstrating the cell count data (lactic acid) obtained by simulating the specimen 3 (high) using the specimen 1 (middle) of the floating cells as the reference data, where the x-axis represents day and the y-axis represents mM/day. FIG. 29 is a graphical illustration demonstrating the cell count data (oxygen) obtained by simulating the specimen 3 (high) using the specimen 1 (middle) of the floating cells as the reference data, where the x-axis represents day and the y-axis represents mM/day. FIG. 30 is a graphical illustration demonstrating the cell count data (carbon dioxide) obtained by simulating the specimen 3 (high) using the specimen 1 (middle) of the floating cells as the reference data, where the x-axis represents day and the y-axis represents mM/day.

In each of FIGS. 27 to 30, the dash-dot line reflects the reference data. The reference data is the cell count data (e.g., first cell count data) obtained by the cell culture of the specimen 1. In each of FIGS. 27 to 30, the broken line reflects actual data. The actual data is the cell count data obtained by the cell culture of the specimen 3. In each of FIGS. 27 to 30, the solid line reflects data (e.g., third cell count data) obtained by combining the actual data and the prediction data.

In each of FIGS. 27–30, the solid line substantially fell within ±5%×day of the broken line, indicating the present inventors concluded that the third cell count data was appropriately generated regarding the specimen 3 of the floating cells.

1. A data processing apparatus for a cell culture apparatus having a hollow fiber membrane, the data processing apparatus comprising:
a calculation unit configured to calculates a rate ratio between:
a first change rate, the first change rate being a change rate of a first cell count data in a first period, the first cell data indicating a change in a first number of cells from a first start of a first culture using the cell culture apparatus to an end of the first culture using the cell culture apparatus under a first culture condition; and
a second change rate, the second change rate being a change rate of a second cell count data in a second period, the second cell count data indicating a change in a second number of cells from a second start of a second culture using the cell culture apparatus to a present time of the second culture using the cell culture apparatus under a second culture condition; and
a prediction data generation unit configured to generate prediction data that indicates a change in the second number of cells after the present time under the second culture condition using a first partial data, the first partial data including a part after the first period of the first cell count data and the rate ratio.

2. The data processing apparatus of claim 1, wherein
the first period is a first predetermined period, and
the second period is a second predetermined period.

3. The data processing apparatus of claim 1, wherein the second period is the same duration as the first period.

4. The data processing apparatus of claim 1, further comprising:
an acquisition unit configured to acquire the first cell count data and the second cell count data.

5. The data processing apparatus of claim 1, further comprising:
a display control unit configured to control a display unit of a cell culturing apparatus based on a third number of cells calculated from a third cell count data, the third cell count data obtained by combining the prediction data and the second cell count data.

6. The data processing apparatus of claim 1, wherein the prediction data generation unit is configured to generates the prediction data when there is a deviation between the second change rate and the first change rate.

7. The data processing apparatus of claim 6, wherein the deviation exists when the second change rate is larger than the first change rate.

8. The data processing apparatus of claim 6, wherein the deviation exists when a difference between the second change rate and the first change rate is a greater than or equal to a predetermined or selected amount.

9. The data processing apparatus of claim 1, wherein the first cell count data includes:
data obtained by previously performed cell culture or cell cultures using the using the cell culture apparatus;
data obtained from one or more simulations of cell culture or cell cultures using the cell culture apparatus; or
a combination thereof.

10. A method for data processing in a cell culture apparatus having a hollow fiber membrane, the method comprising:
calculating a rate ratio that is a ratio between:
a first change rate, the first change rate being a change rate of a first cell count data in a first period, the first cell data indicating a change in a first number of cells from a first start of a first culture using the cell culture apparatus to an end of the first culture using the cell culture apparatus under a first culture condition; and
a second change rate, the second change rate being a change rate of the second cell count data in a second period, the second cell count data indicating a change in a second number of cells from a second start of a second culture using the cell culture apparatus to a present time of the second culture using the culture apparatus under a second culture condition; and
generating prediction data that indicates a change in the second number of cells after the present time using a first partial data, the first partial data including a part after the first period of the first cell count data and the rate ratio.

11. The method of claim 10, wherein
the first period is a first predetermined period, and
the second period is a second predetermined period.

12. The method of claim 10, wherein the second period is the same duration as the first period.

13. The method of claim 10, further comprising:
acquiring the first cell count data and the second cell count data.

14. The method of claim 13, wherein the first cell count data and the second cell count data is acquired by an acquisition unit of a data processing apparatus in communication with the cell culture apparatus.

15. The method of claim 14, wherein
the rate ratio is calculated by a calculation unit of the data processing apparatus, and
the prediction data is generated by a prediction data generation unit of the data processing apparatus.

16. The method of claim 10, further comprising:
displaying a third number of cells on a display unit of the cell culturing apparatus, the third number of cells calculated from a third cell count data, the third cell count data obtained by combining the prediction data and the second cell count data.

17. The method of claim 10, wherein prediction data is generated when there is a deviation between the second change rate and the first change rate.

18. The method of claim 17, further comprising:
recognizing a deviation between the second change rate and the first change rate.

19. The method of claim 17, wherein the deviation exists when the second change rate is larger than the first change rate or when a difference between the second change rate and the first change rate is a greater than or equal to a predetermined or selected amount.

20. The method of claim 10, wherein the first cell count data includes:
data obtained by previously performed cell culture or cell cultures using the using the cell culture apparatus;
data obtained from one or more simulations of cell culture or cell cultures using the cell culture apparatus; or
a combination thereof.
